(19) United States
(12) Patent Application Publication
(10) Pub. No.: US 2009/0326673 A1
(43) Pub. Date: Dec. 31, 2009

(54) POLYMERIC MATERIAL

(75) Inventors: John Neil Devine, Lancashire (GB); Irene Sinz, Bregenz (AT)

Correspondence Address: MICHAEL BEST & FRIEDRICH LLP, 100 E WISCONSIN AVENUE, Suite 3300, MILWAUKEE, WI, 53202, US

(73) Assignee: INVIBIO LIMITED, Lancashire (GB)

(21) Appl. No.: 12/447,319

(22) PCT Filed: Oct. 11, 2007
(86) PCT No.: PCT/GB07/03881

§ 371 Date: Jun. 9, 2009

(30) Foreign Application Priority Data
Oct. 25, 2006 (GB) 0621228.6

(51) Int. Cl. A61F 2/30; B65D 85/00

(52) U.S. Cl. 623/23.39; 206/370

(57) ABSTRACT

An assembly comprises first and second parts which bear against one another, wherein said first part comprises a polyetherketoneketone and said second part comprises a polyetherketoneketone. The parts bear against one another such that one or both of the parts may have a tendency to wear and/or produce wear debris by virtue of contact between the parts. The materials used, however, minimise wear and the production of such wear debris.

This invention relates to polymeric materials and particularly, although not exclusively, relates to the use of such materials in assemblies comprising first and second parts which bear against one another. Preferred embodiments relate to the use of polymeric materials for bearing surfaces, for example for reconstructive joints (or other parts) of human bodies.

A wide range of materials has been proposed for use in reconstructive or artificial joints (or other parts) of human bodies, for example for joints or bearing surfaces in the spine; for shoulder or finger joints; and for partial or total hip or knee replacements.

Tribiology International Vol. 31, No. 11, pp 661-667, 1998 (Wang) describes the success of total hip arthroplasty in the second half of the 20^{th} century as owing greatly to the use of ultra-high molecular weight polyethylene as a bearing surface for the acetabular component. Excellent wear is acknowledged when a polyethylene bearing surface is coupled with a metal or ceramic femoral head. However, a problem is acknowledged in that the debris produced by wear of polyethylene may cause adverse biological reaction, leading to bone loss or osteolysis, and, subsequently, the need to undertake revision surgery.

Many joint replacement devices, for example total hip, knee or disc replacement devices consist of a number of individual parts, due in part to the poor mechanical properties of the polymeric component. For example, in total joint replacements, the end plates are typically made of a cobalt chrome molybdenum alloy for mechanical support. The end plates articulate against an UHMWPE core which results in a specific device height due to restrictions on the part thickness of the UHMWPE component, wear of UHMWPE against one or both end plates and the manufacture and precise implantation of three components.

Metal-on-metal articulation joints have been proposed and used with mixed results. Some metal implants may fail in a relatively short time whilst some will last much longer. Such inconsistent performance is, of course, unacceptable. It may stem from difficulties in controlling manufacturing tolerance of a metal-on-metal implant such as clearance, sphericity, surface finish or the quality of the alloy itself. Whilst more recently metal-on-metal hip joints have been designed with tighter tolerances and superior metal characteristics in an attempt to increase the lifetime of such joints, there is some concern over the body's reaction to the metal ions produced by the wear particles and the effects these may have on the body.

Ceramic-on-ceramic joints have been proposed but these require even higher manufacturing precision than metal-on-metal joints because of the inherent brittleness of the ceramic. Furthermore, the brittleness has been known to lead to fracture of a joint (e.g. in the case of a hip joint, femoral head fracture) which can be difficult to treat and/or effect revision surgery.

Thus metal-on-metal and ceramic-on-ceramic joints are much less forgiving in the design and manufacturing areas and more sensitive to surgical techniques compared to polyethylene/metal joints.

Carbon fibre and ULTRAPEK (Trade Mark) (polyetherketoneetherketoneketone) composite materials have been employed in spinal interbody fusion devices and as bone plates.

It is an object of the present invention to address the aforementioned problems.

According to a first aspect of the invention, there is provided an assembly comprising first and second parts which bear against one another, wherein said first part comprises a polyetherketoneketone.

Said first and second parts may bear against one another so that, in use, one or both of the parts may have a tendency to wear and/or produce wear debris by virtue of contact between the parts. Advantageously, however, the materials from which the first and second parts are made may be such that the amount of wear debris produced and the rate of wear is significantly less than for corresponding parts made from other polymeric materials.

Preferably, said second part comprises a polyetherketoneketone.

Preferably, a bearing surface of said first part which comprises polyetherketoneketone contacts a bearing surface of said second part, wherein said bearing surface of said first part comprises (preferably consists essentially of) a first composition which comprises at least 50 wt %, suitably at least 65 wt %, preferably at least 80 wt %, especially at least 95 wt %, of said polyetherketoneketone. In especially preferred embodiments, said first composition includes at least 97 wt %, especially at least 99 wt % polyetherketoneketone. The first composition may consist essentially of polyetherketoneketone.

In embodiments wherein said first composition does not consist essentially of polyetherketoneketone, said polyetherketoneketone may include one or more fillers. Fillers may be fibrous or non-fibrous. Examples of non-fibrous fillers include x-ray contrast materials, for example barium sulphate or other radio dense metals or salts. Non-fibrous fillers may be present in said first composition to a level of 0-10 wt %, suitably 0-5 wt % or even 0-1 wt %. Preferably however said first composition does not include any radio dense metal or salt; and preferably does not include any non-fibrous fillers. Examples of fibrous fillers include discontinuous fibrous fillers. Such fillers may be present in said first composition to a level of 0-40 wt %, 0-30 wt %, 0-20 wt % or 0-10 wt %. An example of a fibrous filler is carbon fibre. However, preferably said first composition does not include carbon fibre. Preferably, said first composition does not include any fibrous fillers.

Said bearing surface of said second part preferably also comprises polyetherketoneketone. Thus, preferably in the assembly, a bearing surface which comprises polyetherketoneketone suitably contacts a bearing surface which comprises polyetherketoneketone.

Preferably, said bearing surface of said second part comprises (preferably consists essentially of) a second composition which comprises at least 50 wt %, suitably at least 65 wt %, preferably at least 80 wt %, especially at least 95 wt % of said polyetherketoneketone. In especially preferred embodiments, said second composition includes at least 97 wt %, especially at least 99 wt % polyetherketoneketone. The second composition may consist essentially of polyetherketoneketone.

In embodiments wherein said second composition does not consist essentially of polyetherketoneketone, said polyetherketoneketone may include one or more fillers. Fillers may be fibrous or non-fibrous. Examples of non-fibrous fillers include x-ray contrast materials, for example barium sulphate or other radio dense metals or salts. Non-fibrous fillers may be present in said second composition to a level of 0-10 wt %, suitably 0-5 wt % or even 0-1 wt %. Preferably however said second composition does not include any radio dense metal or salt; and preferably does not include any non-fibrous fillers. Examples of fibrous fillers include discontinuous fibrous fillers. Such fillers may be present in said second composition to a level of 0-40 wt %, 0-30 wt %, 0-20 wt % or 0-10 wt %. An example of a fibrous filler is carbon fibre. However, preferably said second composition does not include carbon fibre. Preferably, said second composition does not include any fibrous fillers.

Preferably, said first and second compositions have substantially the same composition.

Said first part may include at least 0.5 g, preferably at least 1 g, more preferably at least 5 g of said polyetherketoneketone. Said first part may include less than 5 kg of said polyetherketoneketone. Said bearing surface of said first part may include 0.5 g, preferably at least 1 g, more preferably at least 5 g of said polyetherketoneketone.

Said first part may have a weight of at least 1 g, preferably at least 5 g. The weight may be less than 5 kg.

Said second part may include at least 0.5 g, preferably at least 1 g, more preferably at least 5 g of said polyetherketoneketone. Said second part may include less than 5 kg of said polyetherketoneketone. Said second part may include less than 5 kg of said polyetherketoneketone. Said bearing surface of said second part may include 0.5 g, preferably at least 1 g, more preferably at least 5 g of said polyetherketoneketone.

Said second part may have a weight of at least 1 g, preferably at least 5 g. The weight may be less than 5 kg.

In the assembly, said first part and said second part are preferably movable relative to one another. For example, a bearing surface of one of the parts may be arranged to slide over a bearing surface of the other part. Said first and second parts may be pivotable relative to one another.

Said first and second parts are preferably lubricated in use. For example they may be lubricated by synovial fluid when used in a human body; or lubricated by a lubrication fluid such as an oil, when used in other applications. Many different types of assemblies comprising first and second parts as described may be provided. Preferably, said assembly is for implantation in a human body, suitably to replace a structural element of the human body. Said assembly may be for use in or around the spine, for example in spinal non-fusion technologies; or for use in artificial joints, for example in fingers, hips, knees, shoulders, elbows, toes and ankles.

One of said first or second parts of the assembly may comprise a male element and the other of said first or second parts may comprise a female element wherein said male and female elements bear against one another, suitably with said bearing surfaces which comprise polyetherketoneketone in contact, and said male element is pivotable relative to the female element.

Said first part may be made substantially entirely from polyetherketoneketone. Alternatively, a first part may comprise a material other than polyetherketoneketone but a bearing surface of such a first part may comprise, preferably consist essentially of, polyetherketoneketone. Such a bearing surface may be defined by capping or coating, or otherwise providing, a layer of polyetherketoneketone on a precursor of said first part for defining said first part. For example, said first part may comprise a metal or ceramic part (e.g. a femoral head) which is capped with polyetherketoneketone or the first part may comprise a natural bearing material such as a natural hard tissue bearing surface for example the glenoid or acetabulum, wherein a bearing surface is capped or otherwise resurfaced with polyetherketoneketone.

Said second part may be made substantially entirely from polyetherketoneketone. Alternatively, a second part may comprise a material other than polyetherketoneketone but a bearing surface of such a second part may be defined by polyetherketoneketone. Such a bearing surface may be defined by capping or coating, or otherwise providing, a layer of polyetherketoneketone on a precursor of said second part for defining said second part. For example, said second part may comprise a metal or ceramic part (e.g. a femoral head) which is capped with polyetherketoneketone or the second part may comprise a natural bearing material as described wherein a bearing surface is capped or otherwise resurfaced with polyetherketoneketone.

One of said first or second parts of the assembly may define a head and the other part may define a socket within which the head is pivotable.

In a preferred embodiment, said assembly may be for a hip replacement. It may comprise a femoral head and an acetabular component. Bearing surfaces which contact one another suitably are defined by, and preferably consist essentially of polyetherketoneketone.

Said polyetherketoneketone suitably includes a repeat unit of formula

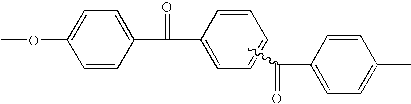

Said polyetherketoneketone may be amorphous or semi-crystalline.

Said polyetherketoneketone is preferably semi-crystalline. The level and extent of crystallinity in a polymer is preferably measured by wide angle X-ray diffraction (also referred to as Wide Angle X-ray Scattering or WAXS), for example as described by Blundell and Osborn (Polymer 24, 953, 1983). Alternatively, crystallinity may be assessed by Differential Scanning Calerimetry (DSC).

The level of crystallinity in said polyetherketoneketone may be at least 1%, suitably at least 3%, preferably at least 5% and more preferably at least 10%. In especially preferred embodiments, the crystallinity may be greater than 30%, more preferably greater than 40%, especially greater than 45%.

Said polyetherketoneketone preferably includes at least 60 mole %, more preferably at least 90 mole % of repeat units of formula I. Preferably, said polyetherketoneketone consists essentially of repeat units of formula I.

Said polyetherketoneketone suitably has a melt viscosity (MV) of at least 0.09 kNsm⁻², preferably at least 0.14 kNsm⁻², more preferably at least 0.35 kNsm⁻².

MV is suitably measured using capillary rheometry operating at 400° C. at a shear rate of 1000 s⁻¹ using a tungsten carbide die, 0.5×3.175 mm.

Said polyetherketoneketone may have a tensile strength, measured in accordance with ASTM D638 of at least 40 MPa, preferably at least 60 MPa, more preferably at least 80 MPa. The tensile strength is preferably in the range 80-120 MPa, more preferably in the range 85-115 MPa.

Said polyetherketoneketone may have a flexural strength, measured in accordance with ASTM D570 of at least 130 MPa. The flexural strength is preferably in the range 130-200 MPa.

Said polyetherketoneketone may have a tensile modulus, measured in accordance with ASTM D638, of at least 2 GPa, preferably at least 3 GPa, more preferably at least 3.3 GPa. The tensile modulus is preferably in the range 3.4-4.5 GPa.

Preferably, said first part and said second part comprise the same material; that is both preferably comprise polyetherketoneketone as described.

Preferably, respective surfaces of said first and second parts which bear against one another have substantially the same composition.

The assembly of the first aspect may include one or more additional parts which may bear against said first and/or said second parts. Said one or more additional parts may comprise polyetherketoneketone as described.

According to a second aspect of the invention, there is provided a kit for providing an assembly of said first aspect, the kit comprising:
(a) a first part as described according to said first aspect;
(b) a second part as described according to said first aspect;
   wherein said first part and said second part are cooperable to define an assembly wherein said first and second parts bear against one another.

Said first part and said second part may have any feature of the first part and the second part of the first aspect mutatis mutandis.

According to a third aspect of the invention, there is provided a package, which is preferably substantially sterile, which comprises an assembly or kit according to the first or second aspects respectively.

According to a fourth aspect, there is provided a method of manufacturing a first part according to the first and second aspects, the method comprising forming a bearing surface of said first part from a polyetherketoneketone.

The method may comprise forming a bearing surface of a second part arranged to be assembled with said first part, wherein said bearing surface comprises polyetherketoneketone.

The method may comprise making one or both of said parts substantially entirely from polyetherketoneketone; or the method may comprise forming one or both bearing surfaces (but not the entirety) of said first and second parts substantially entirely from polyetherketoneketone.

According to a fifth aspect of the invention, there is provided a method of making an assembly according to the first aspect, the method comprising:
(a) selecting a first part as described according to the first aspect;
(b) selecting a second part as described according to the first aspect; and
(c) contacting the first and second parts so that the parts bear against one another and define said assembly.

According to a sixth aspect of the invention, there is provided the use of a first part as described according to the first aspect and a second part as described according to the first aspect in the manufacture of an assembly which comprises said first and second parts bearing against one another for implantation into the human body, for example to replace a structural element of the body.

Any feature of any aspect of the invention or embodiment described herein may be combined with any other feature of any aspect of an invention or embodiment described herein mutatis mutandis.

Specific embodiments of the invention will now be described by way of example.

Pin-on-plate testing was used to assess materials. The pins and plates were made according to the general procedures described in Example 1 and 2 and tested using the general procedure described in Example 3.

### EXAMPLE 1

### General Procedure for Making Pins

All pins were machined from injection moulded plaques. All plaques were produced using standard conditions, for example those described in general literature available from Invibio Limited. The machined pins were polished to give a surface roughness (Sa) of approximately 1 micron. All pins were cleaned in aqueous ethanol and demineralised water and annealed using a general annealing protocol for example as described in general literature available from Invibio Ltd. All pins were machined such that any fibre alignment caused by the direction of polymer flow would be parallel with the reciprocating motion. Unless specified, all pins were gamma sterilised with an irradiation dose of 50 kGy.

### EXAMPLE 2

### General Procedure for Making Plates

All plates were machined from injection moulded plaques. All plaques were produced using standard conditions. The machined plates were machined to maintain the injection moulded surface finish (Sa of approximately 0.1 micron). All plates were cleaned in aqueous ethanol and demineralised water and annealed using a general annealing protocol. All plates were machined such that any fibre alignment caused by the direction of polymer flow would be parallel with the reciprocating motion.

### EXAMPLE 3

### General Procedure for Testing Materials

A pin-on-plate machine was used. The machine was a four station pin-on-plate machine which applied both reciprocation and rotational motion. The reciprocation was applied by a sledge moving along two fixed parallel hardened steel bars and a heated bed, lubricant tray and plate holder were positioned on top of this sledge. The rotational motion was applied to each pin using a small motor. The cycle frequencies of both the reciprocation and the rotation was set at approximately 1 Hz. The plate holder consisted of four wells into which the plate specimens were clamped. A lubricant was contained within the lubricant tray and heated to a temperature of 37° C. by resistors within the bed. This was controlled by a thermocouple. A load (either of 20 N or 40 N) as applied to each station via a lever arm mechanism. A lubricant level sensor made from platinum wire was attached to the lubricant tray to allow the lubricant to be maintained at an almost constant level. This was topped up from a reservoir of distilled water. An electronic counter was connected to the reciprocating sledge. Stroke length was set to 25 mm. A cover was placed over the entire rig to prevent dust contamination from the atmosphere.

The lubricant used was 24.5% bovine serum (protein content: 15 gl⁻¹) with 0.2% sodium azide added to retard the growth of bacteria and 20 mM EDTA to prevent calcium deposition.

The wear was assessed gravimetrically. At least twice a week (approx. 0.25 million cycles) the machine was stopped to allow for cleaning and weighing of the samples. Any excess lubricant was cleaned from the lubricant baths and the pins and plates removed. The samples were then cleaned and dried using a predetermined and consistent protocol. The pins and plates were then weighed three times on a balance (accurate to 0.1 mg) and an average weight recorded. Control specimens were used to take account of the lubricant absorption of both the pins and plates during the test duration. The machine was then reassembled and the lubricant refreshed. The wear tests were performed up to two million cycles.

Vacuum oven drying tests were also performed both before and after the wear tests in an attempt to get the ‘true’ weight loss of these materials and compare this to the standard weight loss measurements. For these tests, in case the vacuum oven drying technique affected the wear properties of the materials, only two sets of samples were analysed. As this drying technique will affect the lubricant absorption throughout the wear test, additional soak controls were also dried in the vacuum oven.

The wear volumes were plotted against sliding distance and the gradient of the line through the data (determined by linear regression analysis) provided the wear rate. The wear rate was then divided by the load and sliding distance to determine the wear factor, k (mm³N⁻¹ m⁻¹).

Using the procedures described in Examples 1 and 2 pins and plates were made and combinations tested under specified loads, using the general procedure described in

### EXAMPLE 3

### Samples of Polyetherketoneketone Bearing Against Itself were Found to Have Surprisingly Good Wear, Characteristics

It should be appreciated that the provision of an articulating device which comprises bearing parts made out of a single type of material (i.e. PEKK) in the absence of other fillers such as carbon fibres may reduce costs, secondary processing steps and the risk of the filler such as carbon fibres being removed during abrasive wear and then acting as a third body wear particle between the bearing parts.

In comparison with metal or ceramic components PEKK materials can be manufactured by a lower cost and more efficient manufacturing route such as injection moulding. There may be additional benefits in using these lower modulus materials compared with metals or ceramics, which can cause stress shielding and subsequent bone resorption.

Other advantages of the materials described are that they are less brittle than ceramics; and use of the materials avoids the production of metallic wear debris and the associated health risk of metal ions being released into the body

An all PEKK prosthesis may allow for a more iso-elastic implant which may advantageously reduce the micromotion at the bone—prosthesis interface compared with a stiffer metal or ceramic part.

Further advantages of the composite materials described include lower weight than metals or ceramics; and improved mechanical properties compared with UHMWPE thereby allowing thinner parts, a greater degree of motion and design flexibility. For example, in joint replacement devices such as total disc replacement devices, the number and/or size of parts may advantageously be reduced.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. An assembly comprising first and second parts which bear against one another, wherein said first part comprises a polyetherketoneketone.

2. An assembly according to claim 1, wherein said first and second parts bear against one another so that, in use, one or both of the parts may have a tendency to wear and/or produce wear debris by virtue of contact between the parts.

3. An assembly according to claim 1, wherein said second part comprises a polyetherketoneketone.

4. An assembly according to claim 1, wherein a bearing surface of said first part which contacts a bearing surface of said second part consists essentially of a first composition which comprises at least 80 wt % polyetherketoneketone.

5. An assembly according to claim 4, wherein said first composition consists essentially of polyetherketoneketone.

6. An assembly according to claim 4, wherein a or said bearing surface of said second part consists essentially of a second composition which comprises at least 80 wt % polyetherketoneketone.

7. An assembly according to claim 6, wherein said second composition consists essentially of polyetherketoneketone.

8. An assembly according to claim 1, wherein said first part includes at least 0.5 grams of polyetherketoneketone, said second part includes at least 0.5 grams of polyetherketoneketone, said first part includes less than 5 kg of polyetherketoneketone and said second part includes less than 5 kg of polyetherketoneketone.

9. An assembly according to claim 1, wherein said first part and said second part are moveable relative to one another.

10. An assembly according to claim 1, wherein said assembly is for implantation in a human body to replace a structural element of the human body.

11. An assembly according to claim 1, wherein said first part is made substantially entirely from polyetherketoneketone and said second part is made substantially entirely from polyetheretherketone.

12. An assembly according to claim 1, wherein one of said first or second parts of said assembly may define a head and the other part may define a socket within which the head is pivotable.

13. An assembly according to claim 1, wherein said first part and said second part comprise the same material.

14. A kit for providing an assembly according to claim 1, the kit comprising:
(a) a first part as described according to claim 1;
(b) a second part as described according to claim 1;
wherein said first part and said second part are cooperable to define an assembly wherein said first and second parts bear against one another.

15. A package, which comprises an assembly or kit according to claim 1.

16. A method of manufacturing a first part according to claim 1, the method comprising forming a bearing surface of said first part from a polyetherketoneketone.

17. A method of making an assembly according to claim 1, the method comprising:
(a) selecting a first part as described according to claim 1;
(b) selecting a second part as described according to claim 1; and
(c) contacting the first and second parts so that the parts bear against one another and define said assembly.

18. The use of a first part as described according to claim 1, and a second part as described according to claim 1 in the manufacture of an assembly which comprises said first and second parts bearing against one another for implantation into the human body.
